(19) United States
(12) Patent Application Publication
(10) Pub. No.: US 2009/0197930 A1
(43) Pub. Date: Aug. 6, 2009

(54) Eyedrops

(75) Inventor: Kenji Yoshida, Tokyo (JP)

Correspondence Address: BIRCH STEWART KOLASCH & BIRCH, PO BOX 747, FALLS CHURCH, VA, 22040-0747, US

(73) Assignee: Kenji Yoshida, Tokyo (JP)

(21) Appl. No.: 12/223,526

(22) PCT Filed: Feb. 2, 2007
(86) PCT No.: PCT/JP2007/052312

§ 371 Date: Dec. 29, 2008

(30) Foreign Application Priority Data
Feb. 2, 2006 (JP) 2006-026120

(51) Int. Cl. A61K 31/4166; A61P 21/02

(52) U.S. Cl. 514/390

(57) ABSTRACT

It is intended to provide eyedrops which have an ocular tension-lowering effect and a hypotensive effect based on novel function mechanisms. The above-described eyedrops contain a muscle relaxant as the active ingredient and dantrolene sodium is preferably usable as the muscle relaxant employed as the active ingredient. The eyedrops lower the ocular tension via the relaxation of the extraocular muscle tension and lower the blood pressure via the relaxation of the sympathetic nerve tension, thereby exhibiting the ocular tension-lowering effect and the hypotensive effect.

### TECHNICAL FIELD

The present invention relates to eyedrops. More particularly, the invention relates to eyedrops useful for prevention and treatment of diseases caused by heightening of ocular tension (glaucoma, ocular hypertension, normal-tension glaucoma, etc.) and hypertension, based on novel function mechanisms.

### BACKGROUND ART

Among diseases caused by heightening of ocular tension, glaucoma is a disease caused by abnormality of visual functions (visual field, visual acuity, etc.) as a result of elevation of ocular tension exceeding normal ranges due to troubles in ocular tension adjusting mechanism. Ocular hypertension is a disease not having abnormality in visual functions in spite of elevation of ocular tension (over 21 mmHg), and this status, when continued, is said to lead to glaucoma. Normal-tension glaucoma is also known, and it is a disease having abnormality in visual functions although the ocular tension is within normal ranges, and in treatment of this disease, it is also attempted to lower the ocular tension. In treatment of these diseases, control of ocular tension is essential.

First, the mechanism of ocular tension variations studied so far is explained. Aqueous chambers (anterior chamber, posterior chamber) are filled with cellular external fluid called aqueous humor. Aqueous humor is a clear liquid derived from serum and produced mainly in ciliary body, and is considered to function as medium for transporting nutrition and wastes of tissues such as lens, ciliary body, iris, and cornea. Hence, the aqueous humor has a circulatory system, in which the aqueous humor is produced in epithelial cells in ciliary process, passes through the gap between the inside of iris and the anterior side of lens and reaches the anterior chamber by way of the pupil, and the majority passes through the trabecular meshwork existing at the angle formed by the inside of cornea and the root of iris, and joins the vein via Schlemm's canal.

The aqueous humor circulates in such circulatory system, and the pressure of this circulating aqueous humor produces the ocular tension. As mentioned above, the aqueous humor is circulating dynamically, and the pressure is not constant, but changes with diurnal variations. This aqueous humor pressure (ocular tension) is determined by (1) aqueous humor production amount, (2) aqueous humor outflow resistance, and (3) venous tension at outflow point. In healthy eyes, the ocular tension varies in a range of 15±5 mmHg with diurnal fluctuation width of about 2 to 4 mmHg. If an abnormal elevation of ocular tension continues due to any cause, blood circulation of optic nerves or nerve axonal flow may be disturbed, and optic nerves may be heavily damaged. This is an outline of pathology of glaucoma.

As mentioned above, since abnormal elevation of ocular tension leads to progress of glaucoma, control of ocular tension is indispensable for treatment and prevention of glaucoma. Recently, the measuring technique of ocular tension is advanced, and along with wide distribution of electronic tonometers, it is not so difficult to find ocular hypertension (abnormal ocular tension). Ocular hypertension must be promptly treated to recover normal ocular tension.

At the present, various ocular hypotensive methods have been proposed, including eyedrops, internal medicine, injection, and surgical technique, but these methods are intended to lower the ocular tension by controlling the circulation of aqueous humor, and the effects are obtained either by decreasing the aqueous humor production, or lowering the aqueous humor outflow resistance.

Typical ocular hypotensive drugs presently used clinically are introduced below.

### (1) Sympathetic Nerve Blockers

Sympathetic nerve blockers show an ocular tension lowering effect by suppressing aqueous humor production from epithelial cells of ciliary process. Sympathetic nerve blocker is sub-classified into α1, α2, and β1, β2, and various types of eyedrops are developed depending on which receptor should be blocked. Generally, β blockers are used widely because of low side effects. Examples: timolol maleate (β blocker), carteolol hydrochloride (β blocker), betaxolol hydrochloride (β1 selective blocker), nipradilol (α, β, blocker), levobunolol hydrochloride (α1, β blocker).

### (2) Prostaglandins

Prostaglandins increase aqueous humor outflow from uveal sclera outflow path by acting on prostaglandin receptor. When used together with sympathetic nerve block, the ocular tension is lowered effectively. However, combined use of prostaglandin eyedrops and sympathetic nerve blocker eyedrops is known to increase the risk of causing damages on the cornea. Examples: isopropyl unoprostone, latanoprost.

### (3) Carbonic Anhydrase Inhibitors

Carbonic anhydrase inhibitors lower the ocular tension by suppressing aqueous humor production by inhibiting the carbonic anhydrase in the ciliary body. Examples: dorzolamide hydrochloride.

### (4) Sympathetic Nerve Stimulants

It is said that sympathetic nerve stimulants lower the ocular tension by lowering the aqueous humor production and aqueous humor outflow resistance, but the detail is unknown. Extremely strong in side effects, and the ocular tension may be raised in a worst case, and the hypotensive effect is not sufficient as compared with the sympathetic nerve blockers, and it is not used widely at the present. Examples: dipivefrin hydrochloride (epinephrine precursor).

### (5) Parasympathetic Nerve Stimulants

Parasympathetic nerve stimulants lower the ocular tension by lowering the outflow resistance of aqueous humor by contracting the pupil (miosis). Like sympathetic serve stimulants, it is strong in side effects and weak in hypotensive action, it is rarely used except for special case such as angle-closure glaucoma. Examples: pilocarpine hydrochloride.

As mentioned above, various medicines have been used, and many new medicines are presently proposed as ocular hypotensive drugs (see, for example, Japanese Patent Application Laid-Open No. 2004-26710).

In the medicines used hitherto, eyedrops containing sympathetic nerve blockers, in particular β blockers have been widely used because they are low in side effects and easy to use. Historically, these eyedrops intended to lower the ocular tension have been developed only for the purpose of suppressing the aqueous humor production. Hence, when ocular hypotensive eyedrops having similar action mechanism (to suppress aqueous humor production) are used simultaneously, it was hard to obtain additional or synergistic effects. Besides, in the eyedrops intended to lower the ocular tension by suppressing the aqueous humor production, rapid lowering of ocular tension is difficult, and sufficient ocular hypotensive effect is not always obtained. Accordingly, not very rarely, the final choice was surgical technique.

In the light of these problems, the present inventor intensively studied ocular hypotensive agents based on action mechanism different from the conventional ocular hypotensive agents. As a result, it was found that the ocular tension is changed not only by fluctuations of aqueous humor circulation, but also by change of tonus of extraocular muscle. The eyeball is supported by extraocular muscles (four rectus muscles and two oblique muscles) in the orbit. When the tonus of the extraocular muscles supporting the eyeball is promoted by various causes, the oppressive stress on the eyeball from the extraocular muscles is increased, and it is found that the ocular tension is increased.

That is, when the extraocular muscles are changed from relaxed state to tense (contracted) state, the eyeball is pulled backward. As a result, soft tissues existing behind the eyeball (retrobulbar cone) receive oppression from the fundus oculi. The retrobulbar cone intensified in tension pushes back the fundus oculi by a similar tension, and the regulating sclera largely expands forward, and the bulbar axis is similarly expanded. By some reason or other, when the extraocular muscle is contracted excessively and such state continues, the internal ocular tension is considered to be increased continuously. Therefore, promotion of ocular tension caused by excessive and continued tonus of extraocular muscles is hardly controlled only by improving the aqueous humor circulation, and it is considered effective to relax or suppress the tonus of extraocular muscles.

On the basis of the above findings, to lower the promoted ocular tension effectively and promptly, it is extremely effective to lower the promoted tonus of extraocular muscles, and the present inventor has discovered that it is effective to use muscle relaxants (in particular, skeletal muscle relaxants) for lowering the tonus of extraocular muscles. It is hence possible to lower the ocular tension on the basis of a completely different action mechanism from the conventional ocular hypotensive agents intended to improve the aqueous humor circulation.

Thus, the present inventor has studied various treating methods of the diseases caused by promotion of ocular tension by lowering the tonus of extraocular muscles, and discovered a novel fact not known so far in the medical field that lowering of tonus of extraocular muscles may lead to decline of tonus of sympathetic nerves, thereby presenting an antihypertensive action. So far, various mechanisms have been disclosed about control of blood pressure. On the basis of understanding of these findings, various drugs have been developed, and control of blood pressure has been attempted. The present discovery by the inventor is a novel blood pressure control mechanism not known so far.

More specifically, it is known that when the sympathetic nerve becomes tense, the tonus of skeletal muscles is intensified in nerve or humor (in hormone). The extraocular muscles surrounding the eyeball and responsible for ocular motion have tissue structure classified as skeletal muscles, and when the tension of sympathetic nerve is intensified, it is known that the tonus of extraocular muscles is intensified like other skeletal muscles. However, it is found that the extraocular muscles have a different individual feature not found in other skeletal muscles. That is, when the tonus of extraocular muscles is lowered, the tonus of sympathetic nerves is lowered, retrogressively to the above phenomenon. It is known that a reflex circuit called oculo-cardiac reflex (or Aschner's reflex) is present between the eye and the heart, and the presently discovered correlation between the tonus of extraocular muscles and the tonus of sympathetic nerves may be possibly related to unknown nerve reflex circuit, and also may be possibly related with the oculo-cardiac reflex. Anyway, by lowering the excessive tonus of extraocular muscles specifically or continuously, it is found that the excessive tonus of sympathetic nerves can be lowered gradually. As a result, it is confirmed that the blood pressure is normalized (lowered).

Interestingly enough, it is found that such reaction does not take place in the absence of excessive tonus of sympathetic nerves or when, if present, the tonus of extraocular muscles is not promoted. That is, in healthy people, there is no adverse effect of excessively lowering the blood pressure. Unlike the known hypotensive agents, increase of pulse rate is not observed as compensational action of lowering of blood pressure. It means that, not acting directly on blood vessels like Ca antagonist, it is a hypotensive action by lowering the tonus of the sympathetic nerves. It is another feature that the blood pressure is lowered effectively in both systolic phase and diastolic phase.

As mentioned before, the eyeball is supported by extraocular muscles (four rectus muscles and two oblique muscles) in the orbit. When the tonus of extraocular muscles supporting the eyeball is promoted by various causes, it is found that promotion of tonus of sympathetic nerves takes place at the same time. It is also known that excessive tonus of sympathetic nerves and its persistence may elevate the blood pressure. On the basis of understanding of these facts, it is extremely effective to lower the promoted tonus of extraocular muscles in order to lower the promoted tonus of sympathetic nerves effectively and promptly, and normalize (lower) the blood pressure. As its method, it is quite rational to relax the tonus of extraocular muscles by dropping the eyedrops containing muscle relaxants (in particular, skeletal muscle relaxants).

### DISCLOSURE OF THE INVENTION

The present invention is based on the above findings, and the invention is intended to present eyedrops containing muscle relaxants as active ingredients, and ocular tension lowering agents or hypotensive agents comprising such eyedrops. As muscle relaxants, skeletal muscle relaxants are preferred, and dantrolene sodium is preferably used as a skeletal muscle relaxant.

The present invention prevents a therapeutic method of hypertension or disease caused by promotion of ocular tension by dropping an effective dose of eyedrops containing muscular relaxants as active ingredients.

The present invention relates to use of muscle relaxants for manufacturing eyedrops having an ocular tension lowering effect or a hypotensive effect.

### BRIEF DESCRIPTION OF THE DRAWINGS

FIG. 1 is a diagram showing changes of ocular tension in subjects in Test Example 1.

FIG. 2 is a diagram showing changes of diastolic pressure in subjects in Test Example 2.

FIG. 3 is a diagram showing changes of systolic pressure in subjects in Test Example 2.

FIG. 4 is a diagram showing changes of heart rate per minute (bpm) in subjects in Test Example 2.

### BEST MODE FOR CARRYING OUT THE INVENTION

Eyedrops of the present invention contain muscle relaxants as active ingredients. In the present invention, muscle relaxants may be any drug capable of relaxing the extraocular muscles (relaxing the tonus). The muscle relaxants are not particularly specified as far as the tonus of extraocular muscles can be relaxed, and examples include skeletal muscle relaxant, Ca antagonist, and anti-choline agent. Commercial medicines may be used as such drugs.

As the muscle relaxant, a skeletal muscle relaxant is preferred, usable examples include dantrolene sodium, baclofen, botulinum toxin, tubocurarine, and suxamethonium. Various skeletal muscle relaxants may be used for lowering the tonus of extraocular muscles, and medicines having action only on extraocular muscles (skeletal muscles) and hardly acting on intraocular muscles (smooth muscles) may be preferred. From such viewpoint, dantrolene sodium having actions of suppressing release of Ca⁺⁺ from the muscular follicles of skeletal muscles, inhibiting Ca⁺⁺ bond on troponin, and suppressing contraction of skeletal muscles may be considered to be one of the ideal medicines as the muscle relaxant to be used for the purpose of relaxing the tonus of extraocular muscles.

The eyedrops of the present invention can be manufactured by mixing the muscle relaxant with a common pharmaceutical carrier, and processing according to a conventional pharmaceutical method. The eyedrops include eye ointments.

The contents of active ingredients in the eyedrops are generally 0.001% to 2.000% (w/v, same hereinafter), and preferably adjusted to about 0.002% to 1.000%, more preferably 0.005% to 0.2%, and most preferably 0.01% to 0.1%.

In manufacturing process of eyedrops, various additives commonly used in this field can be added. Examples include preservatives such as benzalkonium chloride or cetylpyridium chloride, buffers such as borax, boric acid or potassium dihydrogenphosphate, thickeners such as methyl cellulose, carboxy methyl cellulose, polyethylene glycol or chondroitin sulfate, and dissolution aids such as polysorbate. As required, additionally, an isotonic agent may be added to prepare an isotonic solution, and the liquid is adjusted to about pH 6 to 8. Further, a sustained-release agent may be added.

The eyedrops of the present invention may be administered in proper routes depending on the dosage form, and the dose is adjusted properly. In the case of eyedrops, the dose and the method may be adjusted properly depending on the symptom, body weight and age of the patient, and the medicine may be administered once to five times daily, using about 1 to 3 drops every time.

The eyedrops of the present invention are useful for treatment and prevention of various diseases, and are capable of lessening or curing the symptoms by controlling (lowering) the ocular tension, and such diseases include ocular hypertension, glaucoma, and normal-tension glaucoma. Also the eyedrops of the present invention are useful for prevention and treatment of hypertension.

### INDUSTRIAL APPLICABILITY

The eyedrops of the present invention are excellent in ocular tension lowering effect, and are useful for prevention and treatment of ocular hypertension, glaucoma, and normal-tension glaucoma. In particular, since the medicine lowers the ocular tension on the basis of an action mechanism completely different from the conventional ocular tension lowering action based on improvement of aqueous humor circulation, and by using together with the conventional ocular tension lowering agents, synergistic or additional effects may be expressed, and the ocular tension can be lowered promptly and effectively.

The eyedrops of the present invention have a blood pressure lowering action, and can lower the diastolic pressure and systolic pressure effectively, and are very useful as hypotensive agents. In particular, by local action on extraocular muscles, the blood pressure can be lowered, and unlike the conventional hypotensive agents, it is almost free from side effects caused by general administration. In healthy subjects, the blood pressure is not lowed excessively, and unlike the conventional hypotensive agents, as compensational action of hypotension, increase of heart rate is not observed.

### EXAMPLE

The present invention is more specifically described below by showing Example and Test Examples, but the present invention is not limited to these Examples alone.

### Example 1

### Preparation of Eyedrops

Dantrolene sodium (trade name: Dantrium injection, manufactured by Astellas Pharma Inc.) was dissolved in brine, and the active ingredient content was adjusted to 0.01% (hereinafter referred to as Eyedrops 1). By a similar method, eyedrops of active ingredient content of 0.1% were prepared (hereinafter referred to as Eyedrops 2).

### Test Example 1

### Ocular Tension Lowering Test

By obtaining prior consent, in one eye of five subjects (cases 1 to 5), two drops of Eyedrops 1 prepared in Example 1 were administered. After the first administration, 5 minutes later, eyedrops were administered by the same manner as second administration. After the second administration, the ocular tension was measured in every 10 minutes by using pneumatic applanation electronic tonometer (manufactured by Topcon).

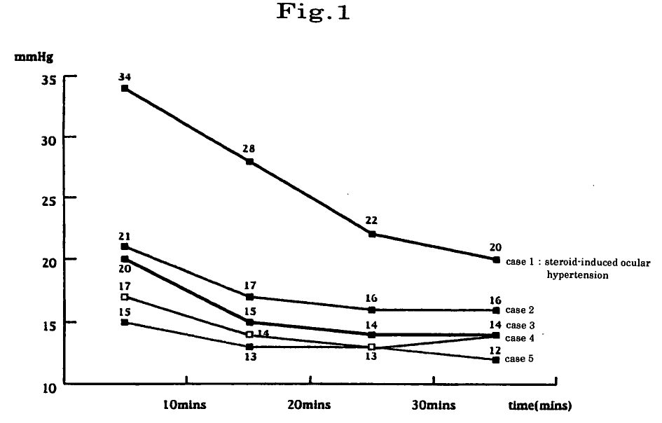

Results are shown in FIG. 1. As shown in FIG. 1, by administration of the eyedrops of the present invention, the ocular tension was obviously lowered from right after dropping, and it was proved that the ocular tension can be lowered by relaxing the tonus of the extraocular muscles.

### Test Example 2

### Hypotensive Test

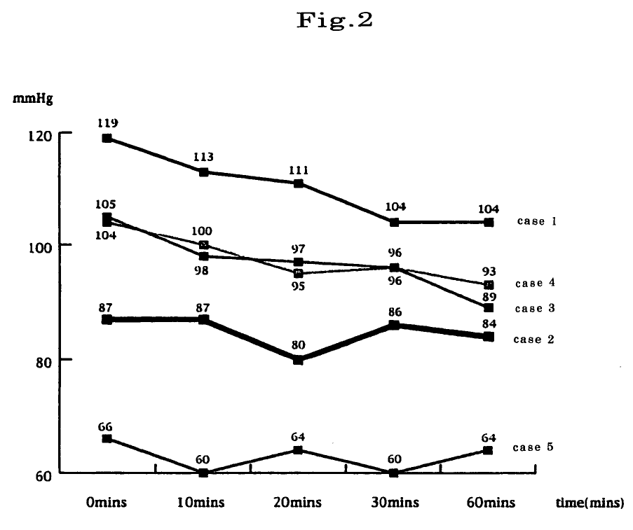

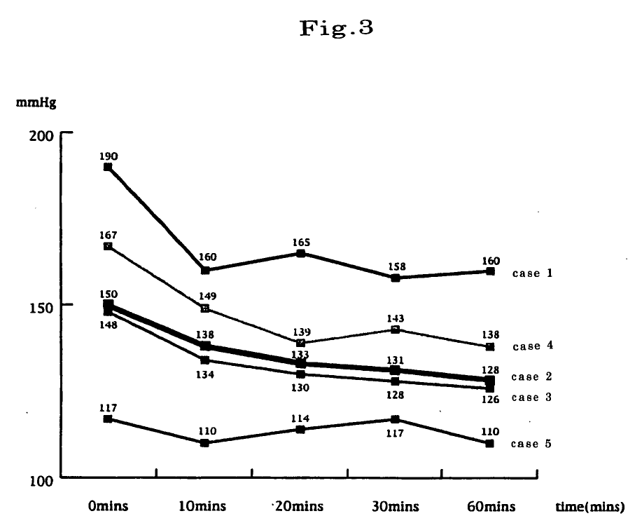

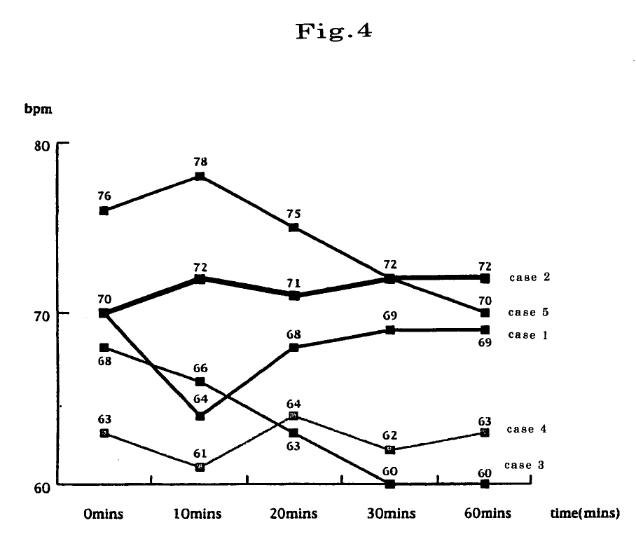

By obtaining prior consent, in both eyes of five subjects (cases 1 to 5), three drops of Eyedrops 2 prepared in Example 1 were administered three times at intervals of 5 minutes. After the third administration, the diastolic pressure, systolic pressure, and the heart rate were measured at time intervals. Results are shown in FIG. 2 (diastolic pressure), FIG. 3 (systolic pressure), and FIG. 4 (heart rate).

The details of five subjects are as follows.

Case 1: 59 years, male, hypertension, not treated.

Case 2: 45 years, male, hypertension, not treated.

Case 3: 46 years, male, hypertension, not treated.

Case 4: 51 years, male, hypertension, not treated.

Case 5: 46 years, female, healthy volunteer.

As shown in FIG. 2, when the eyedrops of the present invention are administered, in cases 1 to 4, the diastolic pressure was lowered slowly. In the healthy volunteer (case 5), the blood pressure was not changed.

As shown in FIG. 3, when the eyedrops of the present invention are administered, in cases 1 to 4, the systolic pressure was lowered remarkably. In the healthy volunteer (case 5), the blood pressure was not changed.

As shown in FIG. 4, the heart rate was not changed notably in any one of cases 1 to 5.

Actually, when the eyedrops of the present invention (dantrolene eyedrops) are administered, a slight myopic inclination and promotion of pupillary motion are observed. Since the majority of pupillary motion is governed by the pupillary sphincter, when the function of pupillary sphincter or ciliary body is slightly promoted, the pupillary reaction or function of pupil or ciliary body tends to be promoted. When the function of pupillary sphincter is lowered, the pupil tends to dilate, and the pupillary reaction such as light reflex becomes dull. Interestingly enough, by administration of dantrolene eyedrops, when the tonus of extraocular muscles is lowered, the pupillary reaction is improved. That is, it teaches that there is a negative correlation between promotion of tonus of extraocular muscles and the tonus of pupillary sphincter and ciliary body. When dantrolene is administered at high concentration, a strong miosis occurs, the pupillary reaction becomes dull, and the myopic inclination is stronger. These changes are same as symptoms observed by dropping parasympathetic nerve active agents such as pilocarpine and acetylcholine. Such reaction of pupillary sphincter is estimated to be caused by excitation of short ciliary nerve. That is, when the tonus of extraocular muscles is lowered artificially, this information is fed back to the ciliary ganglion, and induces a kind of ocular reflex which causes excitation of short ciliary nerve. As a result, the function of pupillary sphincter and ciliary body is promoted (improved).

Thus, when the eyes are observed when administering the eyedrops of the present invention, it is found that relaxation of extraocular muscles causes feedback to the eyes through the nervous system. This local feedback occurring in the eyes is a feedback of short loop mainly passing through the ciliary ganglion, but effects on blood pressure seem to be caused by feedback of long loop mainly passing through the central nervous system and vagus nerve through efferent path.

1. Eyedrops containing a muscle relaxant as an active ingredient.

2. The eyedrops of claim 1, wherein the eyedrops preparation is an ocular tension lowering agent or a hypotensive agent.

3. The eyedrops of claim 1 or 2, wherein the muscle relaxant is a skeletal muscle relaxant.

4. The eyedrops of claim 3, wherein the skeletal muscle relaxant is dantrolene sodium.

5. A method of treating a disease caused by promotion of ocular tension by dropping an effective amount of eyedrops containing a muscle relaxant as an active ingredient.

6. The method of claim 5, wherein the disease caused by promotion of ocular tension is glaucoma, ocular hypertension, or normal-tension glaucoma.

7. The method of claim 5 or 6, wherein the muscle relaxant is dantrolene sodium.

8. A method of treating hypertension by dropping an effective amount of eyedrops containing a muscle relaxant as an active ingredient.

9. The method of treating of claim 8, wherein the muscle relaxant is dantrolene sodium.

10. Use of muscle relaxant for preparing eyedrops having an ocular tension lowering effect or a hypotensive effect.
